(19) United States
(12) Patent Application Publication
(10) Pub. No.: US 2006/0024327 A1
(43) Pub. Date: Feb. 2, 2006

(54) Composition for controlling blood sugar

(75) Inventor: Chuang Chun Chiueh, Taipei (TW)

Correspondence Address: BACON & THOMAS, PLLC, 625 SLATERS LANE, FOURTH FLOOR, ALEXANDRIA, VA, 22314, US

(73) Assignee: Chuang-Chun Chiueh, Taipei City (TW)

(21) Appl. No.: 11/187,798

(22) Filed: Jul. 25, 2005

(30) Foreign Application Priority Data
Jul. 30, 2004 (TW) 93122970
Jul. 16, 2005 (TW) 94119995

(51) Int. Cl. A61K 36/02; A61K 36/73

(52) U.S. Cl. 424/195.17; 424/765

(57) ABSTRACT

The present invention is related to a composition for controlling blood sugar, which includes effective amount of blue algae and mulberry leaves. It can control blood sugar by means of the blue algae helping blood sugar metabolism and mulberry leaves blocking sugar decomposition and entering into blood. The present invention also discloses an oral prophylactic agent containing the composition.

### BACKGROUND OF THE INVENTION

1. Field of the Invention

The present invention is related to a composition for controlling blood sugar, which comprises of effective amount of blue algae and leaves of white mulberries.

2. Description of the Prior Art

Glucose Tolerance Factor (hereafter referring to GTF) which generally exists in all tissues of human body mainly functions as maintaining normal glucose metabolism by ways of synergistic effect with insulin and insulin receptor to transfer blood glucose into cell. GTF is an essential effecter for insulin activating insulin receptor which has a central element of trivalent state chromium (III) composed with mineral substance, vitamins, amino acid and so on to form a complex.

When insulin transfers glucose which is converted to energy into cells, it must incorporate with GTF and insulin receptor simultaneously to transfer glucose into cells effectively. GTP can increase the bioactivity of insulin, strength the sensitivity of substrate, promote the affinity between insulin and insulin receptor and raise the permeability of cell membrane, to facilitate glucose into cells.

Trivalent state chromium (III) untaken from food can transform to GTF in human body and support glucose normal metabolism. However, long term lacking of trivalent state chromium (III) will decrease the amount of GTP which synthesized in human body. Glucose untaken via digestion system can't effectively enter cells and converted into energy; therefore, high concentrated blood glucose flow through kidney because of lack of thoroughly absorbed and leads to clinical symptom of diabetes which glucose is detected in urine.

According to a scientific literature from Saudi Arabia, dividing 78 type II diabetes to 2 groups, which one group ate yeast chromium everyday (23.3 μg Cr/day) and the other ate CrCl₃ (200 μgCr/day) everyday, the experiments progressed for 8 weeks. Then, patients' blood and urine were collected and progressed biochemical analysis including detecting blood sugar (blood sugar on empty stomach and blood sugar after meals), fructosamine (raised after blood sugar level continues to ascend for 2 weeks, not be affected by diet, and more stable then blood sugar detection) and triglyceride. As a result, eating yeast chromium had better effect than eating CrCl₃. People who eat yeast chromium have lowered their blood sugar on empty stomach, blood sugar after meals, fructosamine value and triglyceride value; moreover, they had higher HDL cholesterol and low drug dependence. Some patients even don't need to use insulin anymore.

In 1997 BHNRC research, 180 type II diabetes were grouped into 3 cluster, and separately administrated with 0,100,500 μg Cr×2/day; patients still maintained pharmaceutical and eating habits. Trial results showed that: (1) the HbAlc level of those taking 1000 μg Cr daily had been reduced after 2 months and the other groups also reduced after 4 months; (2) blood sugar on empty stomach of those taking 1000 μg Cr daily had been lowered obviously after 2 months and 4 months; (3) blood sugar of those taking 1000 μg Cr daily after diet 2 hours later had also been lowered obviously after 2 months and 4 months; (4) insulin value on empty stomach and diet after 2 hours of all groups also decrease apparently; (5) People who uptake 1000 μg Cr daily decrease their total cholesterol of blood plasma after 4 months. The data demonstrate that taking Cr can effectively improve the symptom of type II diabetes.

Blue algae are one kind of simple unicellular algae, living in warm, alkaline fresh water and displaying blue-green color because of chlorophyll and phycocyanin. Their morphology shows a spiral and also called Spirulina. Blue algae is abundant in nutrition such as gamma-linolenic acid (GLA), linoleic acid, arachidonic acid, plentiful vitamin B12, iron, protein, RNA and DNA, chlorophyll and phycocyanin, which found only in Blue algae and can raise the survival rate of rats with liver tumor. Blue algae can help human body elevate immune capacity, lower cholesterol, absorb mineral substance, and sweep away toxins. Besides, Blue algae are alkaline which can regulate acid constitution lead by bad eating habits, and prevent or decrease chronic diseases. In 2001, Parikh et al. proceeded clinical trial with 25 type II diabetes. Patients took 2 g Spirulina everyday lasting for 2 months. The results demonstrated that patients' blood sugar, lipid, TG, total cholesterol and LDL-C (low density lipoprotein cholesterol) are lowered and HDL-C (high density lipoprotein cholesterol) is raised. Therefore, the author considered that supplying Blue algae to type II diabetes can control blood sugar and improve blood fat. Further more, Rodriguez-Hernandez et al. made use of diabetes rats to proceed animal test. 5% SM (Spirulina Maxima) in food can prevent from liver fat. The major function of MS is to lower triglyceride in serum and liver, adequately decrease blood sugar of male rats, moreover, avoid oxidation of lipid, recover HDL (high density lipoprotein) to normal standard and reduce total amount of LDL (low density lipoprotein) and VLDL (very low density lipoprotein).

Besides, it exists a natural an alkaloid-1-Deoxynojirimycin (hereinafter DNJ) in leaves of the mulberry, which is a glucosidase inhibitor for inhibiting the activities of α-glucosidase and α-amylase. The chemical structure of DNJ is similar with D-glucose, which prevents starch from hydrolyzed to glucose to inhibit glucose absorption. Presently, several reports have proved that taking mulberry leaves can lower blood sugar and prevent form diabetes. For example, Bondada et al. proceeded clinical trial with 24 male diabetes aging from 40 to 60. Patients were randomly divided into 2 groups, of which one groups took glibenclamide capsules (5 mg/d) and the other one took mulberry leaves powder capsules (3 g/d), lasting for 30 days. After 30 days, the blood and urine of patients were examined and analyzed. The results demonstrated that compared with glibenclamide, mulberry leaves powder capsules apparently can decrease blood sugar by 27% (P<0.01). Simultaneously, the cholesterol amount in blood is decreased by 12% (P<0.01) Moreover, researchers in Mainland China proceeded research with a lowering blood sugar health food mainly made with mulberry leaves. One person uptook 20 ml everyday for 3 months and was detected blood sugar on empty stomach. As a result, the total effective rate is 94.74%, and the remarkable effective rate is 50.88% (patients' blood sugar were lowered to normal standard).

### SUMMARY OF THE INVENTION

Although the prior art had individually disclosed that inorganic chromium and mulberry leaves can lower blood sugar, inorganic chromium absorption rate is very few in human body, even less than 1%. Organic chromium utility rate can reach 10-25% in human body. Therefore, the present invention mixes high level chromium (organic chromium) blue algae with mulberry leaves to form a composition. The composition can reach the purpose of controlling blood sugar concentration by means of blue algae which help blood sugar be metabolized and mulberry leaves which block glucose decomposition as well as entering blood.

The present invention provides a composition for controlling blood sugar concentration, comprising effective amount of blue algae and mulberry leaves. The blue algae contain chromium at least 50 μg/g. referable, chromium in the blue algae ranges from 50 to 120 μg/g, and more preferably, 100 μg/g.

Specifically, a mixture rate of the blue algae and the mulberry leave in the aforesaid composition is 1:1-1:5 by weight. Preferably, the mixture rate is 1:1.5 by weight.

Another object of the present invention provides a composition for controlling blood sugar, comprising effective amount of blue algae and mulberry leaves; a mixture rate of the blue algae and the mulberry leaves is 1:1-1:5 on weight; and the blue algae contains chromium at least 50 μg/g.

The aforesaid blue alga contains chromium ranging from 50 to 120 μg/g, and preferably, 100 μg/g.

The aforesaid mixture rate of the blue algae and the mulberry leaves is 1:1.5 by weight.

Yet another object of the present invention is to provide an oral prophylactic formulation for controlling blood sugar concentration, comprising effective amount of blue algae and mulberry leaves.

The aforesaid oral prophylactic agent further comprises acceptance carrier, additive or excipient.

The aforesaid blue algae contain chromium at least 50 μg/g. Preferably, chromium in the blue algae ranges from 50 to 120 μg/g, and more preferably, 100 μg/g.

Specifically, a mixture rate of the blue algae and the mulberry leave in the aforesaid composition is 1:1-1:5 by weight. Preferably, the mixture rate is 1:1.5 by weight.

The aforesaid oral prophylactic formulation exists in the form of powder, granule or pill. Moreover, it further packs with capsules to form an oral prophylactic capsule.

### DETAILED DESCRIPTION OF THE INVENTION

The composition for controlling blood sugar in the present invention comprises effective amount of high level chromium blue algae and mulberry leaves and has better effect than individual blue algae or mulberry leaves. Based on needs, the composition according to the present invention may be made into powder, spray, granule, liquid, gel or paste by the process that is known to one skilled in the art. The composition supplied can be food, for example, nutrition food including the composition of the present invention.

An oral prophylactic formulation of controlling blood sugar in the present invention comprises effective amount of blue algae and mulberry leaves can be made into any form, for example, but not limited to powder, spray, granule, liquid, gel or paste by the process that is known to one skilled in the art. The oral prophylactic agent of controlling blood sugar in the present invention can further comprises carrier, additive or excipient.

Said “carrier” herein may contain inert component which does not react substantively with other ingredients. Applicable drμg formulation techniques may refer to standard drug formulation techniques as described in Remington's Pharmaceutical Sciences by Mack Publishing Company, Easton, Pa. Suitable drug carrier includes but not limited to sterilized water, normal saline, bacteriostatic saline (containing approximately 0.9% phenyl alcohol), phosphate-buffered saline, Hank's solution, lactated Ringer's solution and other commonly used pharmaceutical carriers.

The “effective amount” means the dose of the compound that will produce beneficial result in the recipient or expected activity in vivo or in vitro. Taking the example of flu, clinical benefits compared to subjects not receiving the treatment include alleviation of symptoms, mitigation of discomfort, shortening the duration of illness, and accelerated healing. The precise dosage to different subjects will be determined by the disease type, severity or symptoms of disease and individual conditions, such as the health state, age, gender, body weight and drug tolerance of the recipient. People familiar with the field may decide dosage based on the factors described above or other factors.

Said “excipient” herein may have multiple functions and purposes, for example, adding disintegrating agent to disintegrate lozenges into tiny granules in the gastrointestinal tract and facilitate its absorption in the process of making oral lozenge, or adding colorant to make it more pleasing and so on. There are other suitable excipients respectively for non-oral formulations, such as injection, suspension, ointment, suppository and spray. Suitable excipients include but not limited to lactose, mannitol, dextran, glucose, glutamic acid, gelatin, sorbitol, trehalose, sucrose, xylitol, starch, microcrystalline cellulose, methyl cellulose, arabic gum or combinations. The utilizing of excipients is common knowledge in this field.

One embodiment of the present invention is a composition (500 mg/unit) including 200 mg blue algae and 300 mg mulberry leaves. The effective amount of the composition is taking 2-4 units of the composition after meals, at least 10 units, equaling to 2 g blue algae and 3 g mulberry leaves, one day. Chromium amount in blue algae is over 50 μg per gram blue algae. After converting, taking over 100 μg Chromium (III) everyday can transformed into GTF, which helps glucose metabolism. Further coupled with DNJ in mulberry leaves, the composition can inhibit the activity of α-glucosidase and α-amylase to prohibit starch from decomposing to glucose, to block absorbing glucose, and to control blood sugar effectively.

According to the suggestion taking amount each day in U.S., it had better no more than 120 μg chromium everyday. Therefore, if blue algae contain 120 μg chromium per gram, each person had better taking 1 g or less than 1 g blue algae. Moreover, if blue algae contain 60 μg chromium per gram, each person had better taking 2 g or less than 2 g blue algae. It means that the taking amount of blue algae depends on the chromium amount of blue algae.

The following examples are presented in order to more fully illustrate the preferred embodiments of the invention. They should in no way be construed, however, as limiting the broad scope of the invention. While the invention is described and illustrated herein by references to various specific material, procedures and examples, it is understood that the invention is not restricted to the particular material combinations of material, and procedures selected for that purpose. Numerous variations of such details can be implied as will be appreciated by those skilled in the art.

The advantages of the present invention are further depicted with the illustration of examples.

### EXAMPLE 1

### The Composition According to the Present Invention can Control Blood Sugar Effectively

Materials

1. Animal series, sex, number: 6-8 week Sprague-Dawley male rats (purchasing from National Laboratory Animal Center) were introduced. The rat number was 16.

2. High blood sugar animal model: After adapting to the environment, rats were fasting for 24 hours, and administrating STZ injection to induce diabetes. After injecting for one week, blood sugar of rats in empty stomach was measured. If the blood sugar value reached or surpassed 13 mM (230 mg/dL), the high blood sugar animal model has been succeeded.

3. testing sample: The content rate of high level chromium blue algae and the mulberry leaves was 1:1.5 in the composition for controlling blood sugar in the present invention

Experimental Steps:

1. Measurement of Blood Sugar in Empty Stomach

High blood sugar animals fasting 3-5 hours were selected and grouped. An experimental group was administrated 300 mg testing sample, controlling group 1 was administrated normal saline 3 ml, and controlling groups 2 was administrated 300 mg general blue algae. After 4 weeks, blood sugar value in empty stomach were measured, and compared between groups.

2. Measurement of Glucose Tolerance

After high blood sugar model rats were fasted for 3-5 days, they were orally administrated 1 g/kg glucose, and detected blood sugar value of before administration, administration after 30 minuets, and administration after 120 minuets.

The results were shown in table 1. After feeding test sample for 4 weeks, controlling group 1 still kept high blood sugar value in empty stomach, and the glucose tolerance rate wasn't obviously lowered. Because of general blue algae contain no chromium, therefore, Rats in controlling group 2 feeding general blue algae still kept high blood sugar value in empty stomach and high glucose tolerance value. However, compared with controlling groups, the blood sugar value in empty stomach and glucose tolerance value in experimental group that were lowered obviously demonstrated that the composition of the present invention has lowering blood sugar effect and doesn't affect normal blood sugar value in normal rats.

TABLE 1

| | Normal | | Diabetes before feeding | | Diabetes after feeding | |
|---|---|---|---|---|---|---|
| | Blood Sugar | | Blood Sugar | | Blood Sugar | |
| | in empty | Glucose | in empty | Glucose | in empty | Glucose |
| | stomach | tolerance | stomach | tolerance | stomach | tolerance |
| Control group(I): | 30.67 ± 18.77 | 152.75 ± 17.91 | 257.00 ± 7.81 | 684.42 ± 60.50 | 564.00 ± 106.59 | 1193.00 ± 84.88 |
| feeding PBS | | | | | | |
| Control group(I): | 10.00 ± 0.00 | 179.88 ± 38.36 | 325.00 ± 21.21 | 799.38 ± 32.70 | 443.00 ± 14.14 | 1153.50 ± 54.80 |
| feeding general | | | | | | |
| blue algae | | | | | | |
| Experimental | 52.50 ± 21.92 | 154.75 ± 37.12 | 309.00 ± 38.18 | 690.13 ± 92.81 | 80.00 ± 2.83 | 548.88 ± 106.24 |
| group: feeding | | | | | | |
| high level | | | | | | |
| chromium blue algae | | | | | | |

### Other Embodiments

All features disclosed herein may be combined in any form with other methods and replaced by other features with identical, equivalent or similar purpose. Thus except for the part that is specifically emphasized, all features disclosed herein constitute only one embodiment among the numerous equivalent or similar features.

All modifications and alterations to the descriptions disclosed herein made by those skilled in the art without departing from the spirits of the invention and appended claims shall remain within the protected scope and claims of the invention.

What is claimed is:

1. A composition for controlling blood sugar concentration, comprising effective amount of blue algae and mulberry leaves.

2. The composition according to claim 1, wherein said blue algae contains chromium at least 50 μg/g.

3. The composition according to claim 2, wherein said blue algae contains chromium ranging from 50 to 120 μg/g.

4. The composition according to claim 3, wherein said blue algae contains chromium 100 μg/g.

5. The composition according to claim 1, wherein a mixture rate of said blue algae and said mulberry leaves is 1:1-1:5 by weight.

6. The composition according to claim 5, wherein said mixture rate of said blue algae and said mulberry leaves is 1:1.5 by weight.

7. A composition for controlling blood sugar, comprising effective amount of blue algae and mulberry leaves;
wherein a mixture rate of said blue algae and said mulberry leaves is 1:1-1:5 by weight; and
said blue algae contains chromium at least 50 μg/g.

8. The composition according to claim 7, wherein said blue algae contains chromium ranging from 50 to 120 μg/g.

9. The composition according to claim 8, wherein said blue algae contains chromium 100 μg/g.

10. The composition according to claim 7, wherein said mixture rate of said blue algae and said mulberry leaves is 1:1.5 by weight.

11. An oral prophylactic formulation for controlling blood sugar concentration, comprising effective amount of blue algae and mulberry leaves.

12. The oral prophylactic formulation according to claim 11, wherein said agent further comprising acceptance carrier, additive or excipient.

13. The oral prophylactic formulation according to claim 11, wherein said blue algae contains chromium at least 50 μg/g.

14. The oral prophylactic formulation according to claim 13, wherein said blue algae contains chromium ranging from 50 to 120 μg/g.

15. The oral prophylactic formulation according to claim 14, wherein said blue algae contains chromium 100 μg/g.

16. The oral prophylactic agent according to claim 1, wherein a mixture rate of said blue algae and said mulberry leaves is 1:1-1:5 by weight.

17. The oral prophylactic formulation according to claim 16, wherein said mixture rate of said blue algae and said mulberry leaves is 1:1.5 by weight.

18. The oral prophylactic formulation according to claim 11, wherein said agent exists in the form of powder, granule or pill.

19. The oral prophylactic formulation according to claim 18, wherein said agent further packing with capsules to form an oral prophylactic capsule.

20. An oral prophylactic formulation for controlling blood sugar concentration, comprising the composition according to claim 7.
